(19) United States
(12) Patent Application Publication
(10) Pub. No.: US 2021/0161647 A1
(43) Pub. Date: Jun. 3, 2021

(54) TENODESIS FIXATION DEVICE

(71) Applicant: Conmed Corporation, Utica, NY (US)

(72) Inventor: Andrew Kam, Odessa, FL (US)

(73) Assignee: Conmed Corporation, Utica, NY (US)

(21) Appl. No.: 17/265,996

(22) PCT Filed: Aug. 9, 2019
(86) PCT No.: PCT/US19/45924

§ 371 Date: Feb. 4, 2021

Related U.S. Application Data
(60) Provisional application No. 62/717,013, filed on Aug. 10, 2018.

(51) Int. Cl. A61F 2/08

(52) U.S. Cl. CPC A61F 2/0811; A61F 2002/0888; A61F 2002/0858; A61F 2002/0835

(57) ABSTRACT

A surgical fixation device and driver for fixing soft tissue in a desired position relative to bone. The fixation device includes a pronged upper portion having two arms extending proximally therefrom to a top surface of the upper portion. Each of the two arms has a side and the sides are opposing sides. The fixation device also includes a flange extending outwardly around each of the opposing sides. The fixation device additionally has a pronged lower portion having two legs extending distally therefrom with a space between the two legs. The upper portion of the fixation device is configured to attach to a surgical driver. The surgical driver has a handle with a body and a shaft extending distally therefrom. A driver interface at a distal end of the shaft is configured for attachment to the fixation device. The driver additionally includes a rotatable adjustment mechanism within the body.

### CROSS-REFERENCE TO RELATED APPLICATIONS

This application claims priority to U.S. Provisional Patent Application Ser. No. 62/717,013, filed on Aug. 10, 2018 and entitled “Tenodesis Fixation Device.”

### BACKGROUND OF THE INVENTION

### 1. Field of the Invention

The present invention is directed generally to a surgical system and, more particularly, to an implant and driver for fixing soft tissue in a desired position relative to bone.

### 2. Description of Related Art

There are several medical procedures where a surgeon needs to attach soft tissue such as tendons or other soft connective tissue to bone. One common example is a torn rotator cuff, where the supraspinatus tendon has separated from the humerus causing pain and loss of ability to elevate and externally rotate the arm. To repair a torn rotator cuff, typically a surgical procedure is used to suture the torn tendon to the bone using a variety of methods. Some procedures utilize large incisions and involve complete detachment of the deltoid muscle from the acromion. Small diameter holes are made in the bone for passing suture material through the bone to secure the tendon. Such large incision procedures are traumatic, causing prolonged pain and recovery time. Other procedures make small incisions and use arthroscopic techniques to attach sutures using either small diameter holes or a bendable tine. Other injuries requiring similar techniques include biceps tendonitis (e.g., a biceps tenodesis procedure) and a torn ACL. Additionally, there are augmentation procedures, such as Lateral Extraarticular Tenodesis, that require the placement of a bone tunnel adjacent to other bone tunnel(s). In these instances, it is preferable to have shallower anchor devices to prevent convergence of the tunnels.

Currently, there are various staple and anchor devices for attaching soft tissue to bone. However, many of these devices suffer from low pull-out strength, a lack of adequate suture attachment sites, a requirement to tie complicated knots with the sutures, complicated threading routines, a failure to assist the surgeon in positioning soft tissue into contact with bone prior to suturing to maximize bonding of the soft tissue to bone, and an overall difficulty in physically handling the devices during surgery.

Generally, injury to joints such as the shoulder and knee involve the tearing or separation of ligaments from their natural position on the bone. The injury leads to a chronic instability in the joint which requires surgical intervention. Modernly, the surgery involves use of one or more arthroscopic devices. These devices include surgical cannulas through which a camera or surgical device are passed. The arthroscopic methods usually involve reduced trauma to the patient than previous methods and can predict a faster recovery.

In brief, the surgical procedures involve visualization and localization of the damage, preparation of the bone surface, implantation of a soft tissue anchor, and suturing of the tissue to the anchor. By tightly contacting the ligament or other soft tissue to a properly prepared bone surface, the two materials bond during the healing process.

Fixation devices are commonly employed during such surgical procedures to secure the soft tissue to bone. The fixation device, such as an implant, is generally inserted into a prepared bone socket so that suture may extend out of the bone socket for stitching to the soft tissue. Drivers are used to insert and impact the implant in the bone socket. Traditional drivers may cause damage to the implant by the impact force. Further, some drivers do not have a mechanism that can be used to organize and tension suture. Therefore, the surgical procedure can take additional time.

Therefore, there is a need for an implant for securely grabbing onto the soft tissue graft for insertion into the prepared bone socket and a driver for inserting and impacting the implant into the prepared bone socket.

Description of the Related Art Section Disclaimer: To the extent that specific patents/publications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/products are discussed above in this Description of the Related Art Section and/or throughout the application, the descriptions/disclosures of which are all hereby incorporated by reference into this document in their respective entirety(ies).

### SUMMARY OF THE INVENTION

Embodiments of the present invention are directed to a surgical fixation device and driver for fixing soft tissue in a desired position relative to bone. According to one aspect, the present invention is a surgical fixation device. The fixation device includes a pronged upper portion having two or more arms extending proximally therefrom to a top surface of the upper portion. Each of the two or more arms comprises a side of the upper portion. The sides of at least two of the two or more arms are opposing sides. A flange extends outwardly around each of the opposing sides. The fixation device also includes a pronged lower portion having two or more legs extending distally therefrom and there is a space between at least two of the two or more legs.

According to another aspect, the fixation device may include a pronged lower portion having two or more legs extending distally therefrom with a space between at least two of the two or more legs. The fixation device may also include an upper portion having a top surface and two or more sides. At least two of the two or more sides have a first slot extending therebetween. Additionally, the fixation device has a central aperture extending into the top surface of the upper portion and through the lower portion.

According to yet another aspect, the present invention is a surgical driver. The driver has a handle having a body with a shaft extending distally therefrom. A driver interface at a distal end of the shaft is configured for attachment to a fixation device. The driver also includes a rotatable adjustment mechanism within the body of the handle.

These and other aspects of the invention will be apparent from and elucidated with reference to the embodiment(s) described hereinafter.

### BRIEF DESCRIPTION OF THE DRAWINGS

One or more aspects of the present invention are particularly pointed out and distinctly claimed as examples in the claims at the conclusion of the specification. The foregoing and other objects, features, and advantages of the invention are apparent from the following description taken in conjunction with the accompanying drawings in which:

FIG. 1 is a side view schematic representation of a fixation device, according to an embodiment;

FIG. 2 is a top view schematic representation of the fixation device, according to an embodiment

FIG. 3 is a perspective view schematic representation of the fixation device in use in a pre-deployment configuration, according to an embodiment;

FIG. 4 is a perspective view schematic representation of the fixation device in use in a deployed configuration, according to an embodiment;

FIG. 5 is a cross-sectional side view schematic representation of the fixation device in use in the deployed configuration, according to an embodiment;

FIG. 6 is a top view schematic representation of a fixation device, according to an alternative embodiment;

FIG. 7A is a perspective view schematic representation of a press-fit driver interface, according to an embodiment;

FIG. 7B is a perspective view schematic representation of a threaded driver interface, according to an embodiment;

FIG. 7C is a perspective view schematic representation of a slotted driver interface, according to an embodiment;

FIG. 7D is a perspective view schematic representation of a keyed driver interface, according to an embodiment;

FIG. 8 is a side view schematic representation of a fixation device, according to an alternative embodiment;

FIG. 9 is a front view schematic representation of the fixation device of FIG. 8;

FIG. 10 is a top view schematic representation of the fixation device of FIG. 8;

FIG. 11 is a side perspective view schematic representation of a driver, according to an embodiment;

FIG. 12 is a perspective view schematic representation of the driver of FIG. 11 loaded with the fixation device, according to an embodiment;

FIG. 13 is a partial exploded view schematic representation of the adjustment mechanism of the driver of FIG. 11, according to an embodiment;

FIG. 14 is a top perspective view schematic representation of the distal end of the handle of the driver, according to an embodiment;

FIG. 15 is a top view schematic representation of the spooling wheel, according to an embodiment;

FIG. 16 is a side view schematic representation of the spooling wheel, according to an embodiment;

FIG. 17 is a top perspective view schematic representation of the cover on the handle of the driver in a closed position, according to an embodiment;

FIG. 18 is a top perspective view schematic representation of the cover on the handle of the driver of FIG. 17 in an open position, according to an embodiment;

FIG. 19 is a top perspective view schematic representation of the cover on the handle of the driver in a closed position, according to an alternative embodiment; and

FIG. 20 is a top perspective view schematic representation of the cover on the handle of the driver of FIG. 19 in an open position, according to an alternative embodiment.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known structures are omitted so as not to unnecessarily obscure the invention in detail. It should be understood, however, that the detailed description and the specific non-limiting examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

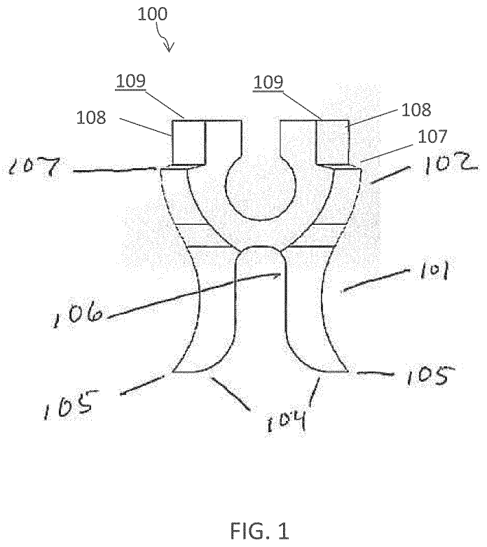

Referring now to the figures, wherein like reference numerals refer to like parts throughout, FIG. 1 shows a side view schematic representation of a fixation device 100, according to an embodiment. The fixation device 100 is a solid surgical implant. The fixation device 100 may be composed of metallic and/or polymeric material. The fixation device 100 comprises an upper portion 102 and a lower portion 101. The upper portion 102 is wider than the lower portion 101 because the lower portion 101 is sized and configured to be inserted into a prepared bone socket.

As shown in FIG. 1, the lower portion 101 is pronged such that a plurality of legs 104 extend therefrom. In the depicted embodiment, the lower portion 101 comprises two legs 104. Each of the plurality of legs 104 terminate in a sharp edge 105. The sharp edges 105 aid the fixation device 100 in grabbing onto the prepared bone socket. As the lower portion 101 is pronged, a space 106 exists between at least two of the plurality of legs 104. The space 106 is sized and configured to accommodate soft tissue.

Still referring to FIG. 1, the upper portion 102 of the fixation device 100 is also pronged such that a plurality of arms 108 extend therefrom. In the depicted embodiment, the upper portion 102 comprises two arms 108. Each of the plurality of arms 108 comprises an outer flange 107. The flange 107 is under a top surface 109 of the upper portion 102. In other words, the flange 107 is between the top surface 109 and the lower portion 101. In an embodiment, the flange 107 is a few millimeters from the top surface 109 to provide fixation under the dense cortical layer of bone.

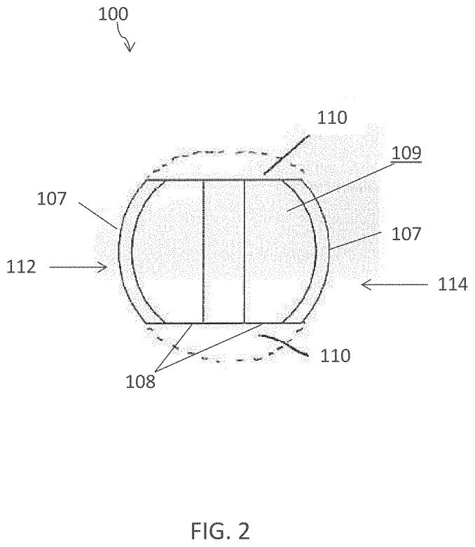

Turning now to FIG. 2, there is shown a top view schematic representation of the fixation device 100, according to an embodiment. As shown in FIG. 2, the flanges 107 extend outward from the arms 108. The fixation device 100 additionally comprises one or more material reliefs 110 in the upper portion 102. In the depicted embodiment, the fixation device 100 comprises two reliefs 110 and the reliefs 110 are on opposing sides 112, 114 of the fixation device 100. With the reliefs 110 on opposing sides 112, 114 of the fixation device 100, soft tissue is allowed to traverse down one side 112, then under the space 106 (FIG. 1), and back up the opposing side 114.

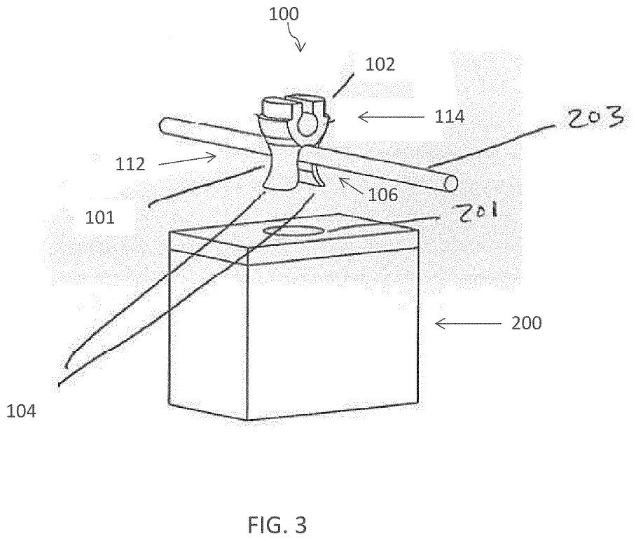

Referring now to FIGS. 3-5, there are shown various perspective and side views schematic representations of the fixation device 100 in use, according to an embodiment. Prior to use of the fixation device 100, an appropriately sized socket 201 is prepared in a bone 200. Thereafter, the fixation device 100 is loaded over soft tissue 203 and positioned above the prepared bone socket 201, as shown in FIG. 3. The soft tissue 203 is within the space 106 between the legs 104 of the lower portion 101 of the fixation device 100. Note, the lower portion 101 has is sized and configured to fit within the prepared bone socket 201. The sides 112, 114 of the upper portion 102 of the fixation device 100 are relieved slightly (via the reliefs 110 in FIG. 2) to allow the fixation device 100 to extend out of the prepared bone socket 201, but provide compression once fixated.

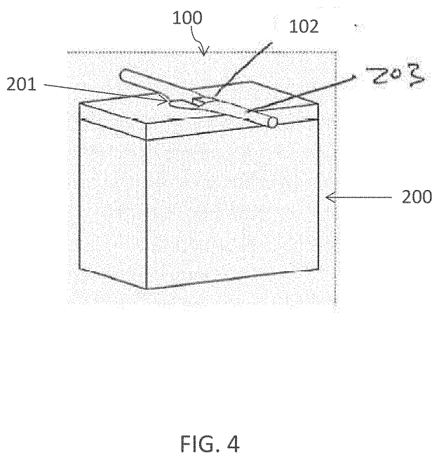

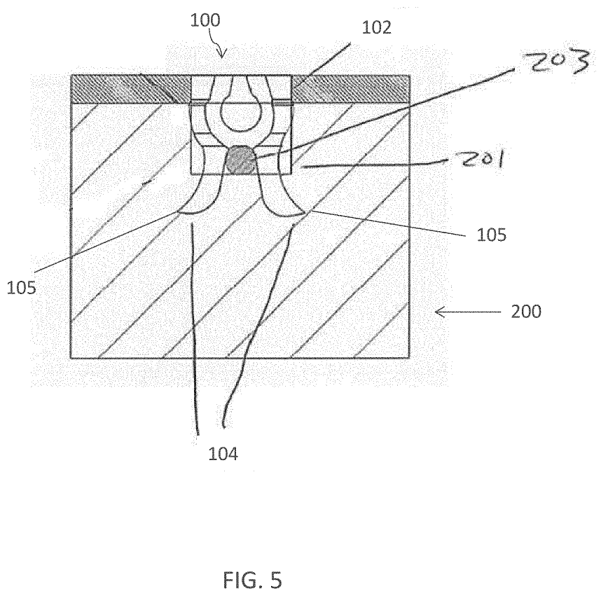

FIG. 4 shows the fixation device 100 impacted into the prepared bone socket 201. As the upper portion 102 of the fixation device 100 is impacted into the prepared bone socket 201, the upper portion 102 is compressed. Compression causes rotation of the legs 104 outwardly about the soft tissue 203 (like a fulcrum), as shown in FIG. 5. Outward deformation of the legs 104 allows for anchoring of the fixation device 100 within the prepared bone socket 201. As described above, the sharp edge 105 of each leg 104 catches on the prepared bone socket 201, aiding in the anchoring.

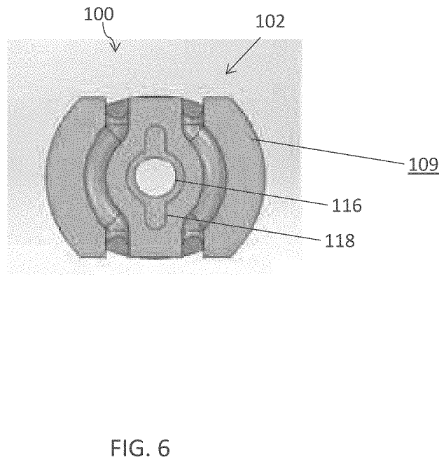

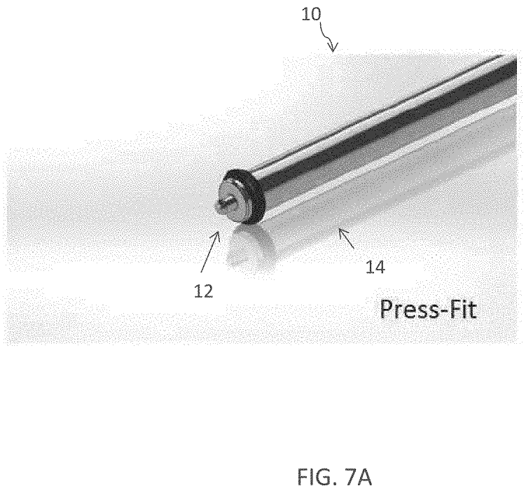

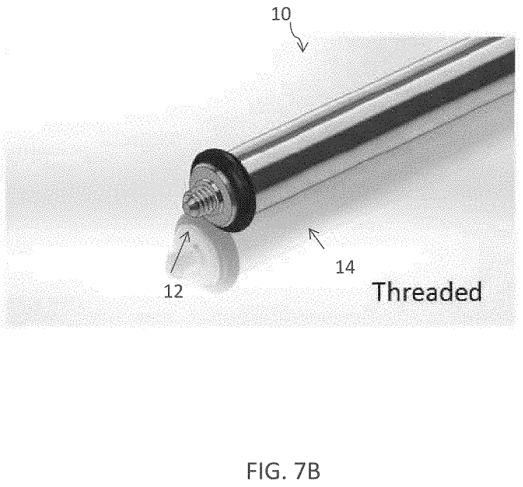

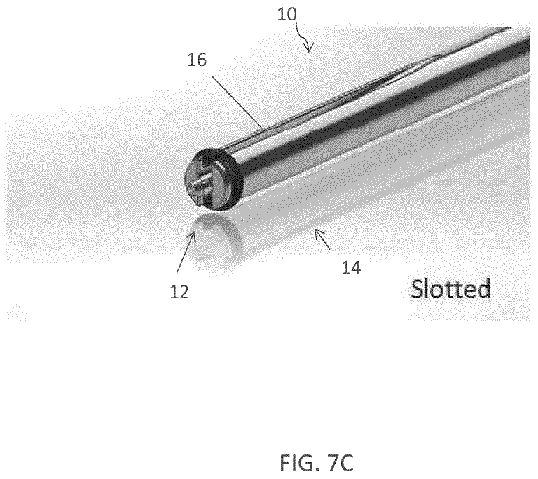

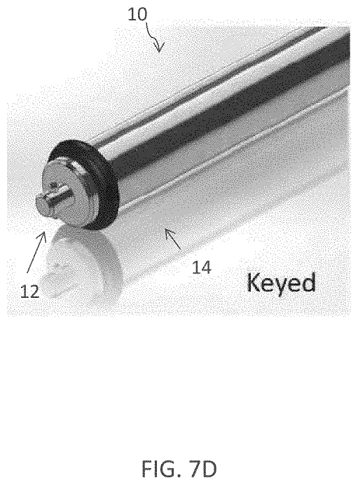

Turning now to FIG. 6, there is shown a top view schematic representation of a fixation device 100, according to an alternative embodiment. In the embodiment of the fixation device 100 shown in FIG. 6, the top surface 109 of the upper portion 102 comprises a connection feature 116 sized and configured to interface with a driver 10 (FIGS. 7A-7D). In one embodiment, the connection feature 116 is an aperture. When the connection feature 116 is an aperture, the fixation device 100 can interface with a variety of driver interfaces 12 at a distal end 14 of the driver 10, as shown in FIGS. 7A-7D.

The driver interface 12 shown in FIG. 7A is a press-fit attachment which is sized and configured to fit within the aperture 116. The driver interface 12 shown in FIG. 7B is a threaded attachment which is also sized and configured to fit within the aperture 116; however, the aperture 116 has complimentary threads (not shown). FIG. 7D shows a driver interface 12 that is a keyed attachment sized and configured to engagement the connection feature 116 shown in FIG. 6. The connection feature 116 shown in FIG. 6 is an aperture 116 and an elongated recess 118 extending across the top surface 109 of the upper portion 102, through the aperture 116. The driver interface 12 in FIG. 7D is sized and configured to fit within the aperture 116 and the elongated recess 118. Turning now to FIG. 7C, the driver interface 12 is slotted or includes a slotted attachment comprising one or more slots 16 extending along the distal end 14 of the driver 10. The driver 10 with slots 16 is preferably used with suture (not shown), as described in detail below.

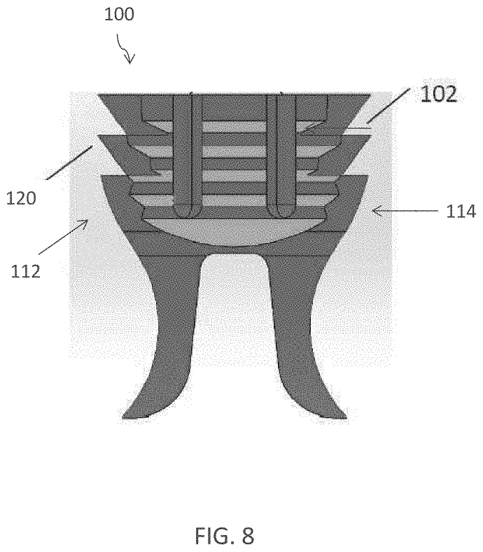

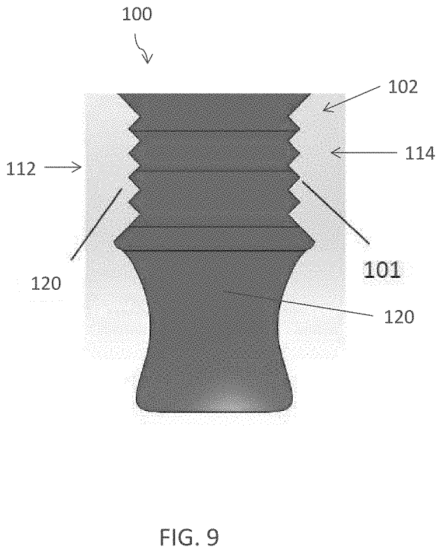

Referring now to FIGS. 8-10, there are shown various view schematic representations of the fixation device 100, according to an alternative embodiment. FIGS. 8 and 9 show side and front views schematic representations of the fixation device 100. The fixation device 100 comprises one or more ribs 120 extending across the sides 112, 114 of the upper portion 102. The ribs 120 increase surface contact area and drag on the fixation device 100. Further, the ribs 120 increase load sharing on the bone and accommodate varying cortical thickness. In the depicted embodiment, at least one of the ribs extends in a plan that is substantially parallel to the top surface 109 of the upper portion 102.

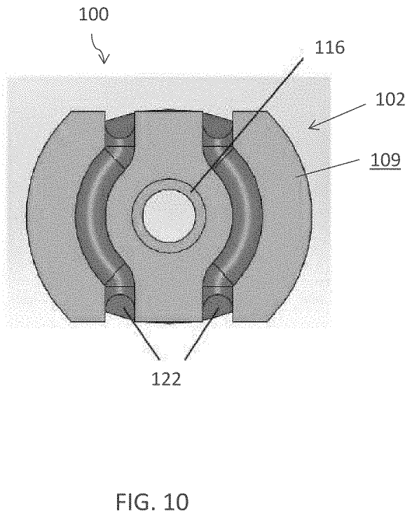

FIG. 10 shows a top view schematic representation of the fixation device 100. The upper portion 102 comprises one or more relief slots 122. In the embodiment shown in FIG. 10, the top surface 109 of the upper portion 102 has two relief slots 122. The two relief slots 122 are on opposing sides of the aperture 116. As shown in FIG. 10, the relief slots 122 are configured radially around the aperture 116 to optimize wall thickness of the driver interface 12, accommodating a larger driver interface 16 (e.g., FIG. 7D). This, in turn, provides sufficient clearance for the inclusion of sutures through the central aperture 116, while maintaining sufficient strength of the driver interface 12.

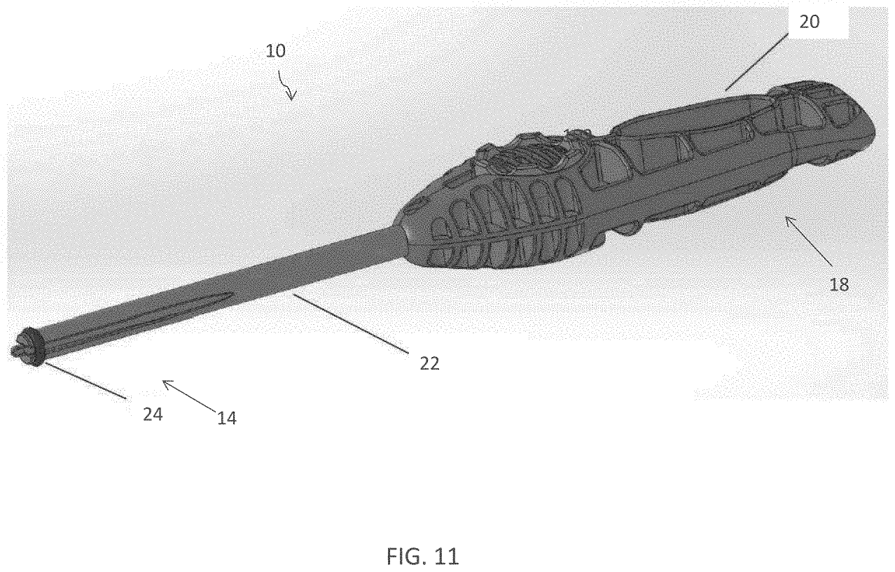

Turning now to FIGS. 11-20, there are shown various views schematic representations of a driver 10 and components thereof, according to an embodiment. FIG. 11 shows a side perspective view schematic representation of a driver 10 sized and configured for impacting the fixation device 100 in a prepared bone socket 201 (FIGS. 3-5) of a defined diameter and depth. The driver 10 comprises a proximal end 18 and a distal end 14. The proximal end 18 comprises a handle 20 connected to a shaft 22 which extends from the handle 20 to the distal end 14. The shaft 22 allows axial impaction for inserting the fixation device 100. The handle 20 can be curved and ergonomically shaped for comfortable use.

As shown in FIG. 11, the shaft 22 extends to a stop 24 at the distal end 14. In the depicted embodiment, the stop 24 is a positive stop sized and configured to be employed at a predetermined location relative to the fixation device 100. The positive stop 24 controls the insertion depth of the fixation device 100 to ensure that the top surface 109 of the fixation device 100 is relatively flush with the outer cortical layer of the bone. As also shown in FIG. 11, the positive stop 24 is comprised of a flexible member, such as an O-ring. In the depicted embodiment, the positive stop 24 is an O-ring with an outer diameter that is greater than an outer diameter of the shaft 22. The O-ring serves as an indicator as to when proper anchor insertion depth has been achieved, while preventing damage to the cortex as the driver 10 is impacted against the bone.

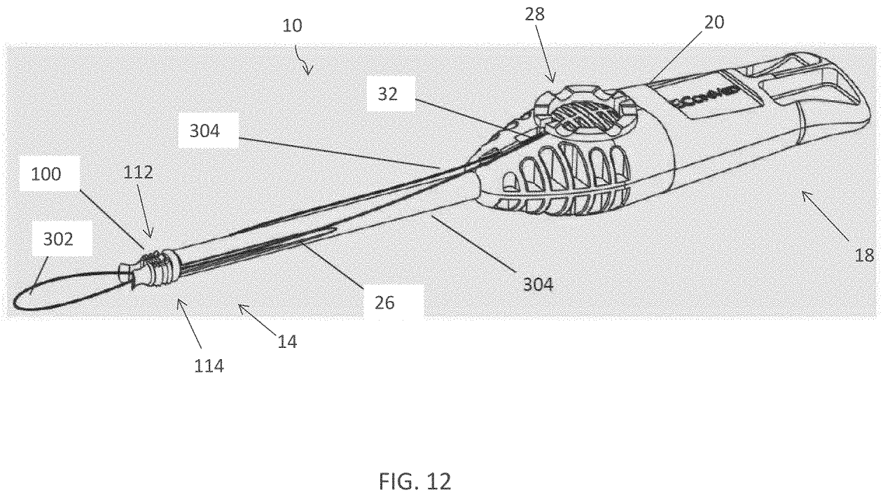

FIG. 12 shows a perspective view schematic representation of a driver 10, according to an alternative embodiment. In the depicted embodiment, the fixation device 100 is attached to the distal end 14 of the driver 10. The fixation device 100 has one or more sutures 300 threaded or otherwise attached thereto for capturing soft tissue, stitching soft tissue to the fixation device 100, or tying a portion of a fixated tendon over the top surface 109 of the upper portion 102 of the fixation device 100. The driver 10 may also comprise one or more slots 26 along at least a portion of the length of the shaft 22 (see slots 16 in FIG. 7D). In FIG. 12, the driver 10 has two slots 26. The two slots 26 extend along at least a portion of the shaft 22 on opposing sides of the driver 10. Specifically, the slots 26 shown in FIG. 12 are substantially aligned with the sides 112, 114 of the fixation device 100.

Still referring to FIG. 12, the slots are sized and configured to receive suture 300. The suture 300 extends from the lower portion 101 of the fixation device 100. In particular, the suture 300 is folded to create a distal loop 302 with two limbs 304, 306 extending proximally therefrom. The distal loop 302 extends from the lower portion 101 and the two limbs 304, 306 extend through the fixation device 100, then out through the aperture 116 (FIG. 10), and exit the driver 10 through the slots 26, as shown in FIG. 12. Thus, the limbs 304, 306 can freely travel within the space 106 in the fixation device 100 and along the shaft 22. In an embodiment, the limbs 304, 306 are collected and tensioned at an adjustment mechanism 28 at the handle 20.

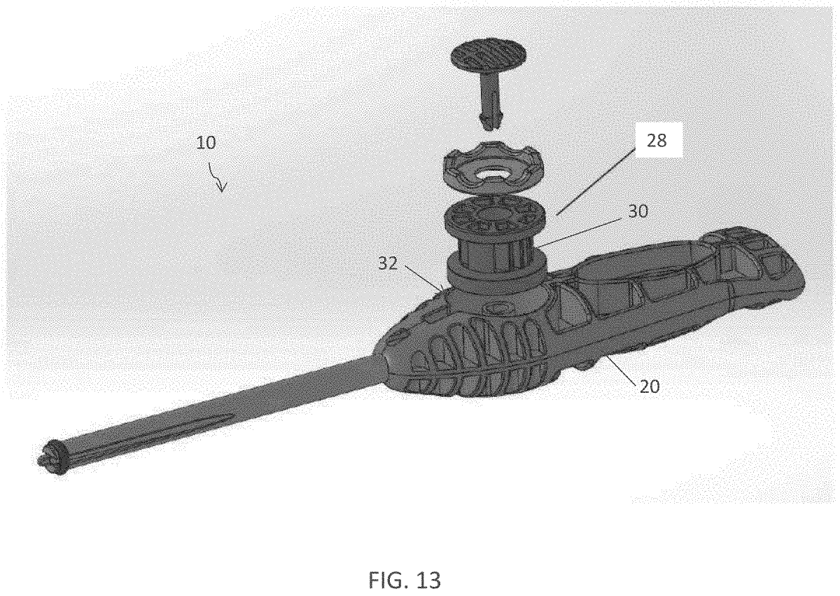

Turning now to FIG. 13, there is shown a partial exploded view schematic representation of the adjustment mechanism 28 of the driver 10, according to an embodiment. The adjustment mechanism 28 in the handle 20 of the driver 10 is rotatable. In particular, the adjustment mechanism 28 is a spooling wheel 30 within a cavity 32 in the handle 20, as shown in FIG. 13. The limbs 304, 306 of suture 300 extend along the shaft 22 and are coupled to the adjustment mechanism 28 (spooling wheel 30), as shown in FIG. 12. The spooling wheel 30 can be rotated clockwise to increase the size of the distal loop 302 and counterclockwise to decrease the size of the distal loop 302 (or vice versa). Thus, the adjustment mechanism 28 serves to adjust the size of the distal loop 302.

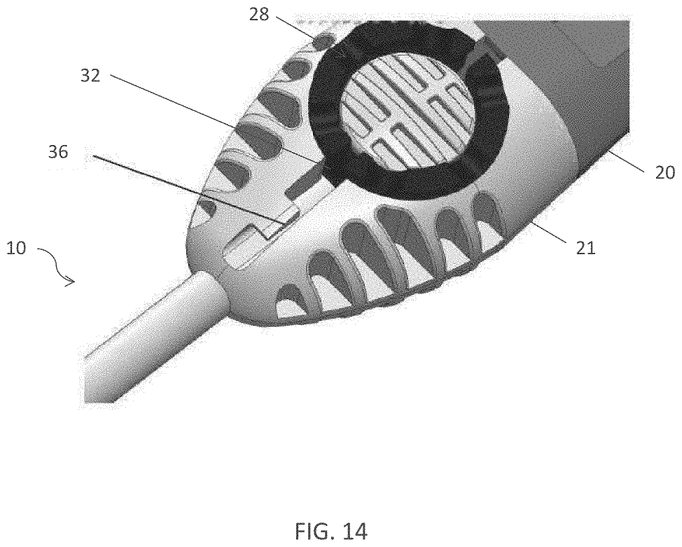

Turning now to FIGS. 14-16, there are shown various views schematic representations of adjustment mechanism 28 of the handle 20, according to an embodiment. FIG. 14 shows a close-up top view schematic representation of the adjustment mechanism 28 of the handle 20. As shown in FIG. 14, a distal end 34 of the handle 20 comprises a slot 36 in a body 21 of the handle 20. The slot 36 begins at or near the distal end 34 of the handle 20 and extends to the adjustment mechanism 28. In some cases, the slot 36 extends to the cavity 32 in the handle 20. The slot 36 allows the suture 300 (and in some cases, needles attached thereto) to separate from the handle 20.

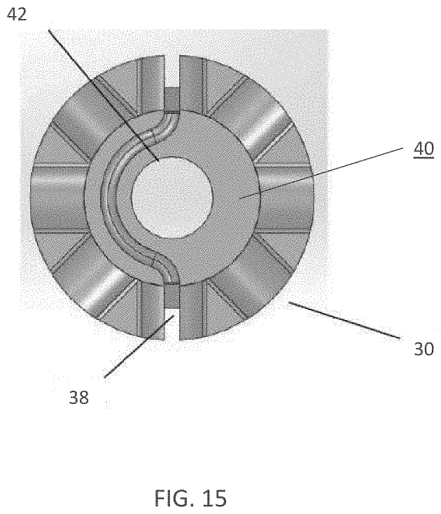

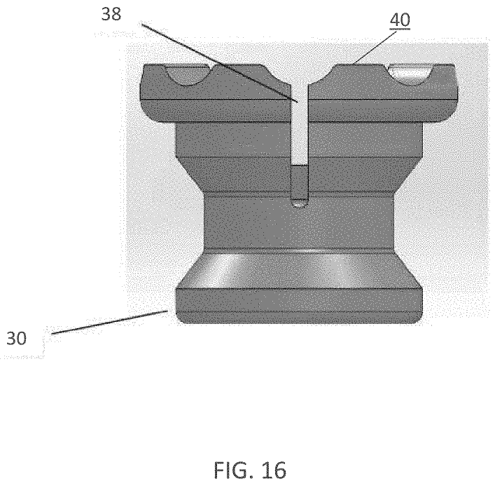

FIG. 15 shows a top view schematic representation of the spooling wheel 30 and FIG. 16 shows a side view schematic representation of the spooling wheel 30. The spooling wheel 30 comprises a slot 38. The slot 38 extends through a top surface 40 of the spooling wheel 30, as shown in FIGS. 15 and 16. The slot 38 allows the suture 300 to travel around a central hub 42 (FIG. 15) of the spooling wheel 30. The slot 38 also allows the suture 300 to wrap around the spooling wheel 30 and release from the spooling wheel 30.

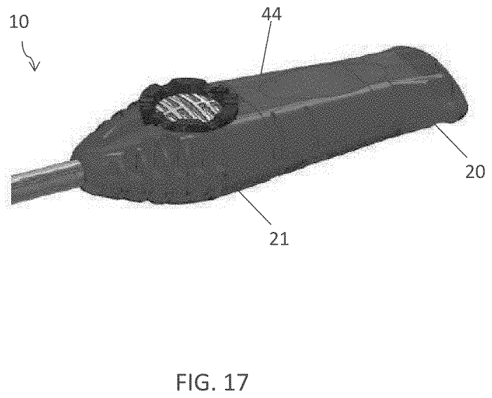

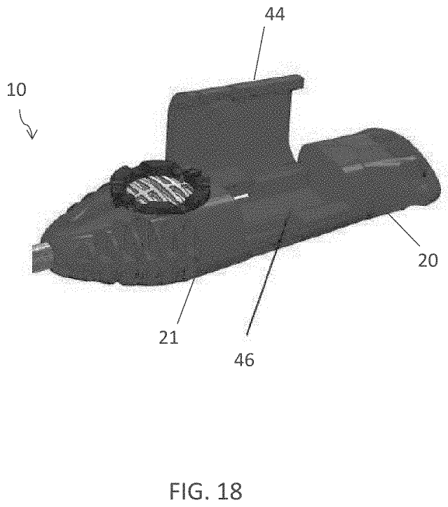

Referring now to FIGS. 17-20, there are shown multiple top perspective views schematic representations of the handle 20 of the driver 10, according to an embodiment. FIGS. 17 and 18 show top perspective views of the handle 20 of the driver 10 having a flip (or rotatable) cover 44. As shown in FIG. 17, the handle 20 comprises a cover 44 that, in a closed position, is flush with a body 21 of the handle 20. In an open position, as shown in FIG. 18, the cover 44 is rotates in a direction away from the body 21 of the handle 20, exposing a compartment 46 within the handle 20. The compartment 46 extends into the body 21 of the handle 20. In the depicted embodiment, the cover 44 is rectangular, although any suitable geometry can be used. The compartment 46 is sized and configured to house or otherwise contain needles (not shown) for stitching suture 300. Needles are often attached to the ends of the limbs 304, 306 of suture 300 to facilitate the ability to stitch the suture 300 to additional soft tissue for fixation to the fixation device 100.

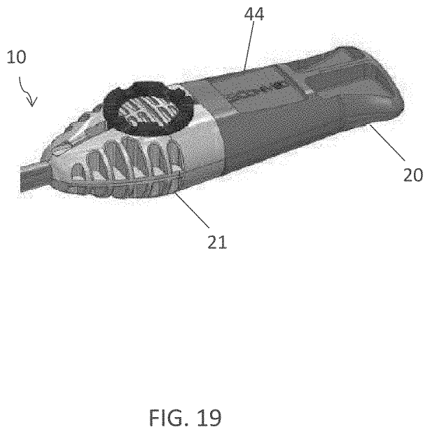

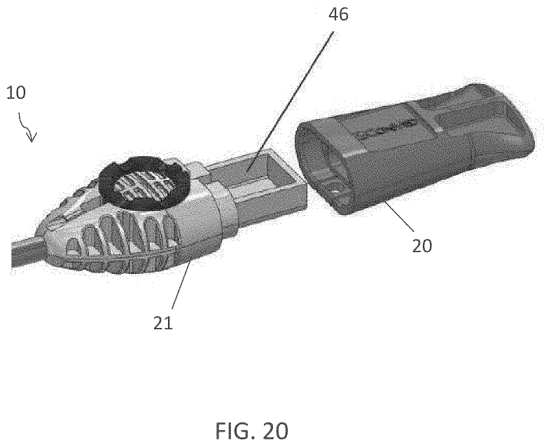

FIGS. 19 and 20 show top perspective views of the handle 20 of the driver 10 having a slide cover 44. As shown in FIG. 19, the handle 20 comprises a proximal cover 44 that, in a closed position, is flush with a body 21 of the handle 20. In an open position, as shown in FIG. 20, the cover 44 slides in the proximal direction away from the body 21 of the handle 20, exposing a compartment 46 within the handle 20. The compartment 46 extends into the body 21 of the handle 20. In the depicted embodiment, the cover 44 is rectangular, although any suitable geometry can be used. The compartment 46 shown in FIGS. 19 and 20 is sized and configured to store needles for the reasons stated above. The compartment 46 is a safe place to store needles as the compartment 46 is away from the user's hand during insertion of the fixation device 100.

All definitions, as defined and used herein, should be understood to control over dictionary definitions, definitions in documents incorporated by reference, and/or ordinary meanings of the defined terms.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as, “has” and “having”), “include” (and any form of include, such as “includes” and “including”), and “contain” (any form of contain, such as “contains” and “containing”) are open-ended linking verbs. As a result, a method or device that “comprises”, “has”, “includes” or “contains” one or more steps or elements. Likewise, a step of method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts and equivalents of all means or step plus function elements in the claims below, if any, are intended to include any structure, material or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the present invention for various embodiments with various modifications as are suited to the particular use contemplated.

What is claimed is:

1. A surgical fixation device, comprising:
a pronged upper portion having two or more arms extending proximally therefrom to a top surface of the upper portion, each of the two or more arms comprising a side of the upper portion;
wherein the sides of at least two of the two or more arms are opposing sides;
a flange extending outwardly around each of the opposing sides; and
a pronged lower portion having two or more legs extending distally therefrom with a space between at least two of the two or more legs.

2. The device of claim 1, wherein the flange is between the top surface and the lower portion.

3. The device of claim 1, wherein each of the two or more legs terminates in a sharp edge.

4. The device of claim 1, wherein the upper portion is wider than the lower portion.

5. The device of claim 1, further comprising a slot extending through the top surface of the upper portion, separating the two of the two or more arms.

6. The device of claim 1, further comprising at least one relief feature on a side of the upper portion adjacent to at least one of the two or more arms.

7. A surgical fixation device, comprising:
a pronged lower portion having two or more legs extending distally therefrom with a space between at least two of the two or more legs;
an upper portion having a top surface and two or more sides;
wherein at least two of the two or more sides having a first slot extending therebetween; and
a central aperture extending into the top surface of the upper portion and through the lower portion.

8. The device of claim 7, further comprising one or more ribs extending around the two or more sides of the upper portion.

9. The device of claim 8, wherein the one or more ribs each extend in a plane substantially parallel to the top surface of the upper portion.

10. The device of claim 7, wherein the first slot extends across the top surface between one of the two or more sides and the central aperture.

11. The device of claim 7, further comprising a second slot extending across the top surface between another of the two or more sides and the central aperture.

12. The device of claim 7, further comprising an elongated recess extending into the top surface of the upper portion and across the central aperture.

13. A surgical driver, comprising:
a handle having a body with a shaft extending distally therefrom;
a driver interface at a distal end of the shaft configured for attachment to a fixation device; and
a rotatable adjustment mechanism within the body of the handle.

14. The surgical driver of claim 13, further comprising one or more slits extending proximally from the distal end of the shaft along at least a portion of the shaft.

15. The surgical driver of claim 13, wherein the adjustment mechanism comprises a spooling wheel within a recess in the body of the handle.

16. The surgical driver of claim 15, further comprising a slot at or near a proximal end of the body of the handle extending to the recess.

17. The surgical driver of claim 15, further comprising a slot extending through a top surface of the spooling wheel.

18. The surgical driver of claim 13, further comprising a compartment within the body of the handle.

19. The surgical driver of claim 18, further comprising a movable cover attached to the body of the handle, wherein in an open position, the cover exposes the compartment and in a closed position, the cover conceals the compartment.

20. The surgical driver of claim 19, wherein the cover is rotatable away from the body of the handle.

21. The surgical driver of claim 19, wherein the cover is slidable along the body of the handle.
